(19) United States
(12) Patent Application Publication
(10) Pub. No.: US 2021/0308311 A1
(43) Pub. Date: Oct. 7, 2021

(54) DECONTAMINATION ROOM AND METHOD OF CONTROLLING DECONTAMINATION

(71) Applicant: Igor, Inc., Johnston, IA (US)

(72) Inventor: Dwight L. Stewart, Johnston, IA (US)

(21) Appl. No.: 17/222,586

(22) Filed: Apr. 5, 2021

Related U.S. Application Data
(60) Provisional application No. 63/004,983, filed on Apr. 3, 2020.
(60) Provisional application No. 63/015,100, filed on Apr. 24, 2020.
(60) Provisional application No. 63/038,465, filed on Jun. 12, 2020.

(51) Int. Cl. A61L 2/24; A61L 9/20; F24F 8/22; A61L 2/10

(52) U.S. Cl. CPC A61L 2/24; A61L 9/20; A61L 2/20; A61L 2/10; F24F 8/22

(57) ABSTRACT

Disclosed are systems and processes that are usable to disinfect a room.

### BACKGROUND

### 1. Field

Example embodiments relate to a decontamination room and a method of controlling decontamination.

### 2. Description of the Related Art

With the onset of COVID 19, decontaminating surfaces has become extremely important. Generally speaking, decontamination may be performed by hand or by specialized equipment and each may destroy a wide variety of bacteria and viruses. While specialized equipment may, in some cases, be preferable to sanitizing by hand, utilizing conventional decontamination equipment is quite difficult and often inflexible.

### SUMMARY

The inventor has noticed that while conventional methods of decontaminating a room are successful, conventional methods are often time consuming, expensive, and imperfect. For example, manual decontamination using sprays often does not cover every surface in a room rendering an occupant thereof vulnerable to contracting bacteria and/or viruses. More sophisticated equipment may improve decontamination, but the sophisticated equipment is often expensive and inflexible, making their use quite difficult. To solve these problems the inventor set out to design a system and method for decontaminating a room which is flexible and easy to use.

### BRIEF DESCRIPTION OF THE DRAWINGS

Example embodiments are described in detail below with reference to the attached drawing figures, wherein:

FIGS. 1A and 1B illustrate an example of a room in accordance with example embodiments;

FIG. 2 illustrates a floor of a building in accordance with example embodiments;

FIG. 3 illustrates the floor associated with virtual spaces in accordance with example embodiments;

FIG. 4 is a view of a controller in accordance with example embodiments;

FIGS. 5-8 are views of databases in accordance with example embodiments;

FIG. 9 is a view of a method in accordance with example embodiments;

FIG. 10 is a view of a system in accordance with example embodiments;

FIG. 11 is a view of a system in accordance with example embodiments; and

FIGS. 12A and 12B are views of an open area in accordance with example embodiments.

### DETAILED DESCRIPTION

Example embodiments will now be described more fully with reference to the accompanying drawings. Example embodiments are not intended to limit the disclosure since the disclosure may be embodied in different forms. Rather, example embodiments are provided so that this disclosure will be thorough and complete, and will fully convey the scope of the disclosure to those skilled in the art. In the drawings, the sizes of components may be exaggerated for clarity.

In this application, when a first element is described as being “on” or “connected to” a second element, the first element may be directly on or directly connected to the second element or may be on or connected to an intervening element that may be present between the first element and the second element. When a first element is described as being “directly on” or “directly connected to” a second element, there are no intervening elements. In this application, the term “and/or” includes any and all combinations of one or more of the associated listed items.

In this application, spatially relative terms merely describe one element's relationship to another. The spatially relative terms are intended to encompass different orientations of the structure. For example, if a first element of a structure is described as being “above” a second element, the term “above” is not meant to limit the disclosure since, if the structure is turned over, the first element would be “beneath” the second element. As such, use of the term “above” is intended to encompass the terms “above” and “below”. The structure may be otherwise oriented (rotated 90 degrees or at other orientations) and the spatially relative descriptors used herein interpreted accordingly.

Example embodiments are illustrated by way of ideal schematic views. However, example embodiments are not intended to be limited by the ideal schematic views since example embodiments may be modified in accordance with manufacturing technologies and/or tolerances.

The subject matter of example embodiments, as disclosed herein, is described with specificity to meet statutory requirements. However, the description itself is not intended to limit the scope of this patent. Rather, the inventors have contemplated that the claimed subject matter might also be embodied in other ways, to include different features or combinations of features similar to the ones described in this document, in conjunction with other technologies. Example embodiments relate to systems and methods for decontaminating a room.

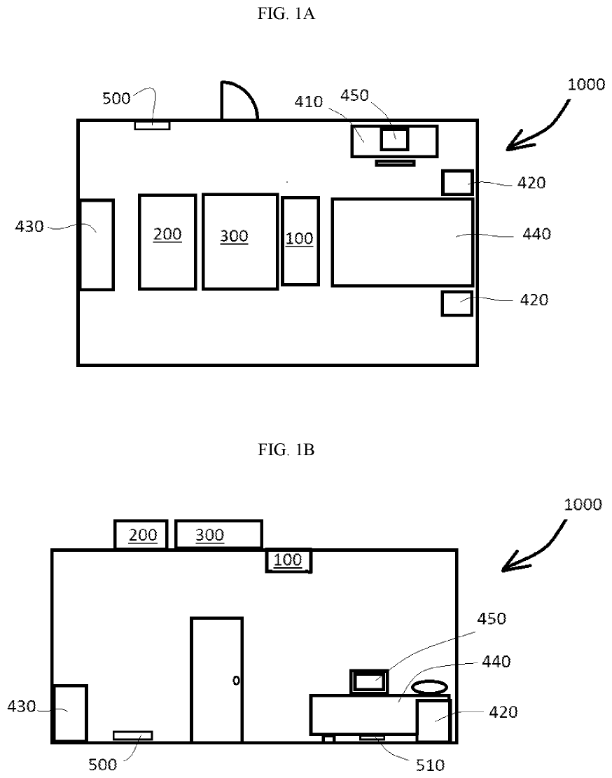

FIGS. 1A and 1B are schematic views of a room 1000 which includes various types of decontamination equipment. For example, the equipment could include, amongst other types of decontamination equipment, a UV light 100, a gas vaporizer 200, and an air purifier 300 (for example, a UV light purifier). The room 1000 may be any kind of room, for example, the room 1000 may be associated with a room in a retirement home, a hospital surgical room, a hospital waiting room, a room in an office building, a clean room in a pharmaceutical plant, or a room in a residential home or apartment. In other words, the room 1000 may represent a multitude of different types of rooms and the type of room is not meant to limit the inventive concepts. The room 1000, of course, may include various types of equipment 400 and structures therein and the equipment and structures 400 may have surfaces requiring disinfecting. For example, the room 1000 may include counter tops, tables, desks 410, filing cabinets, end tables 420, dressers 430, beds 440, and computers 450, all of which may have surfaces that may require decontamination (or at least have surfaces which are desired to be decontaminated). Of course, the rooms are not required to have any of the aforementioned equipment and may have equipment which is not included in the aforementioned list.

In example embodiments room 1000 may also include additional components that may be part of a control system. For example, the room 1000 may include one or more light sensors 500 to detect whether a correct dosage of light has been applied to the room 1000 from a UV light 100 and/or air purifier 300. Other sensors may include chemical sensors 510 which may help determine an amount of a chemical, for example, a disinfectant such as, but not limited to, vaporized hydrogen peroxide, has been applied to the room 1000. Further yet, room 1000 may include other sensors, for example a carbon dioxide sensor 520. The carbon dioxide sensor 520 may measure an amount of carbon dioxide in a room which may indicate how often the air in the room 1000 is or has been rebreathed by people in the room. Data from the carbon dioxide sensor 520 may be used to control ventilation in room 1000. For example, if carbon dioxide in a room exceeds a preset value or if the rate of carbon dioxide accumulation exceeds a preset value, then the room's ventilation system may be activated to bring fresh air into the room. In the alternative, the room 1000 may include an air purifier which may be activated to purify the air if the sensed carbon dioxide level exceeds a preset value or if the rate of carbon dioxide accumulation exceeds a preset value.

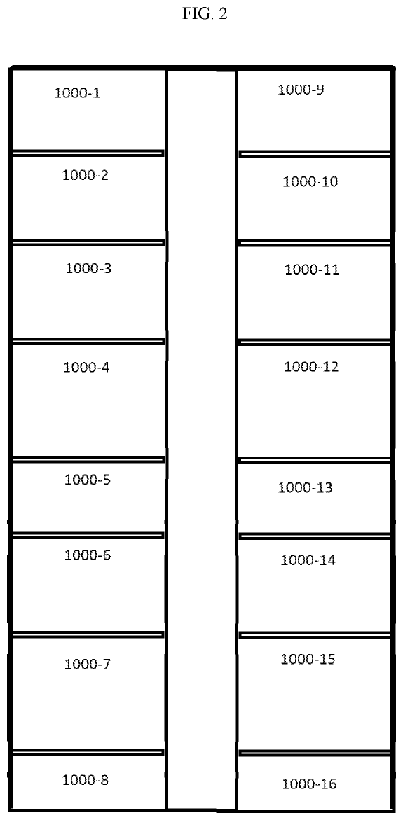

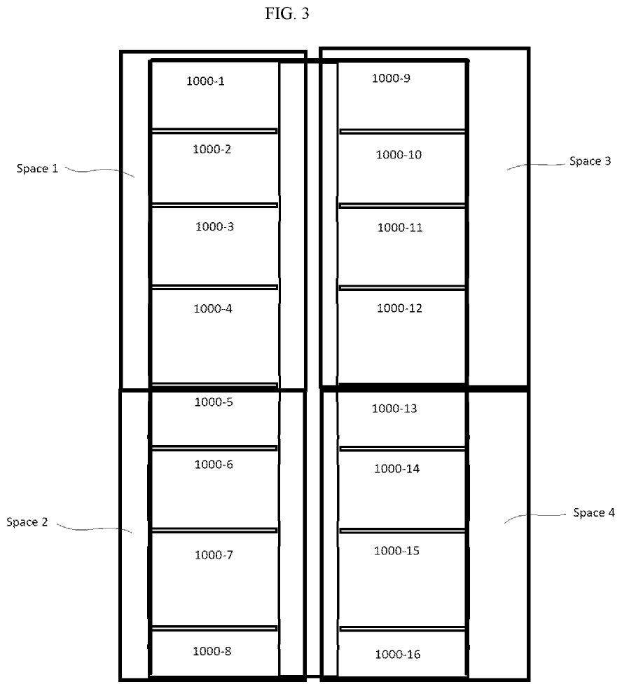

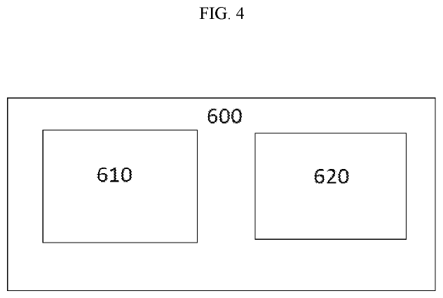

In example embodiments, the room 1000 may be exemplary of a plurality of rooms that may be in a building. For example, FIG. 2 illustrates a floor of a building with sixteen different rooms labeled 1000-1 to 1000-16. Each room may be identical or each room may be different. Some or all rooms may contain some or all of the decontamination equipment previously mentioned or even more decontamination equipment. The rooms 1000-1 to 1000-16 may also be associated with a virtual space that is under the control of a computer 600 (an example of a controller). In fact, the computer 600 may control a plurality of spaces associated with a plurality of rooms which may require disinfecting. For example, FIG. 3 illustrates the previously mentioned floor of rooms and four virtual spaces Space 1, Space 2, Space 3, and Space 4. To that end the computer 600 may include a processor 610 for executing an algorithm or a script and an electronic memory 620 which may associate rooms of a building with a space. In fact, the electronic memory 620 may also associate various types of equipment with a space as well. Though the use of virtual spaces to control one or more rooms has certain advantages (for example, it requires less computer memory), the inventive concepts are not strictly limited to embodiments using virtual spaces and may be implemented without.

In example embodiments, scripts may be stored in the memory 620 to control the various types of decontamination equipment. For example, a first script may be associated with UV light 100, a second script may be associated with gas vaporizer 200, and a third script may be associated with air purifier 300. Although three types of decontamination equipment has been provided, it is understood there could be any number of different types of decontamination equipment present in any given room or space and any number of different scripts to control the decontamination equipment. For example, a fourth script may be associated with controlling a ventilation system using data obtained from carbon dioxide sensor 520.

For purposes of illustration only, the first script may control the brightness level of the UV light 100, whether the UV light 100 strobes, the exposure duration, and a minimum exposure. The first script may also determine whether an appropriate amount of UV light has been provided to the room using data from the sensor 500. The second script may control how long the gas vaporizer 200 operates or how much vaporized decontamination gas will be distributed to a room and/or space. The aforementioned sensor 510 may be used to determine how much decontamination gas has been dispensed to a room and/or a space and the computer 600 may use this data in the script controlling the gas vaporizer 200. The third script may control air purifier 300. It may control, for example, how long the air purifier 300 is operated. The fourth script may control a ventilation fan to bring air into a room based on the data provided to computer 600 by carbon dioxide sensor 520. In example embodiments, the scripts may be executed sequentially or in parallel depending on the preference of an administrator and the scripts may end after the greater than or equal to a set duration and/or the greater than equal to a set minimum dosage target (for example, a set amount of UV light and/or decontamination gas).

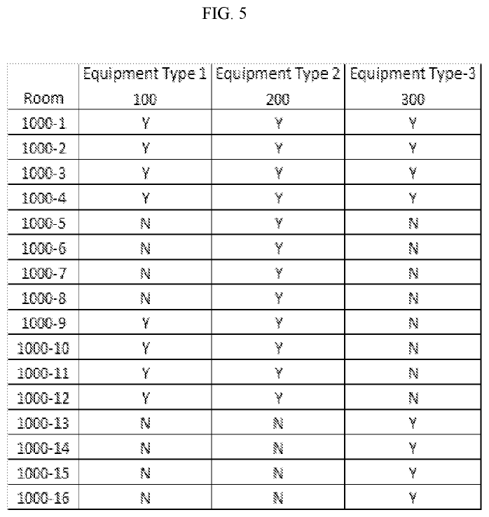

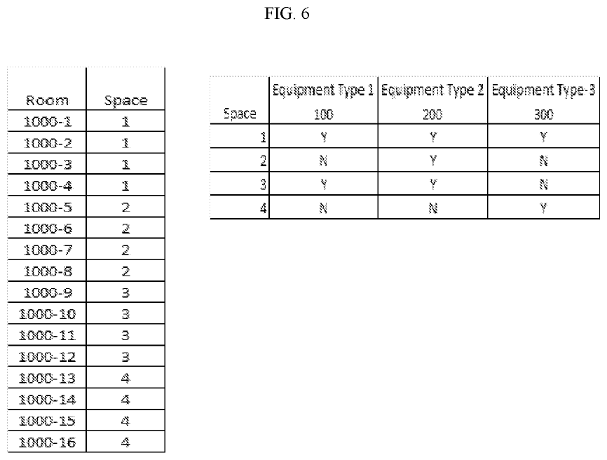

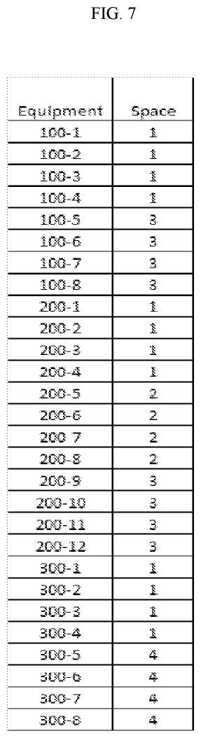

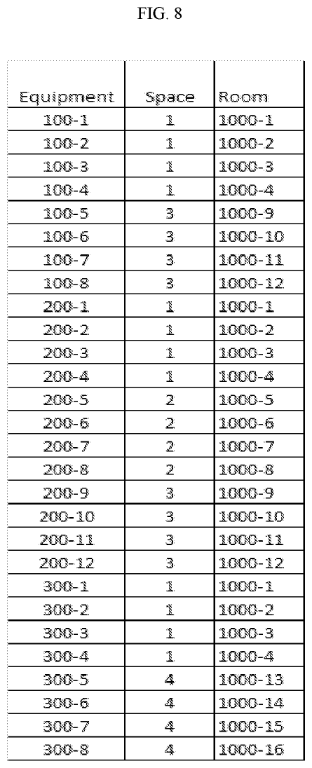

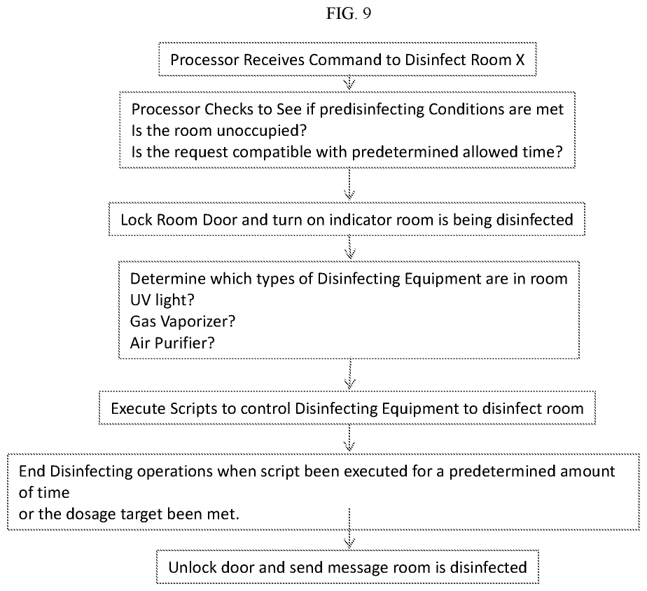

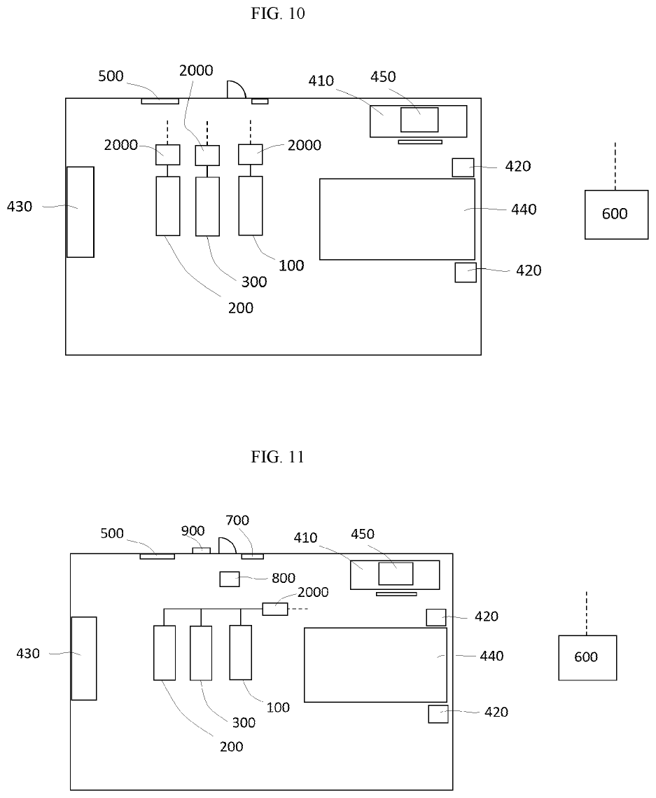

FIG. 5 is a view of an electronic database that may store information regarding each room and the type of decontamination equipment in each room. The Y and N may indicate whether the equipment is present in the room to enable the processor to initiate one or more decontamination operations. The invention, however, is not limited thereto. For example, FIG. 6 illustrates a couple of databases which may be available to the microprocessor 610. The first table associates rooms with spaces and the second associates the types of equipment available in the various spaces. The microprocessor 610 may use the latter tables instead of the former table to control decontamination operations. As yet another example, the database of FIG. 7 may be used. In FIG. 7, the pieces of equipment may be associated with a space. For example, when a piece of equipment is installed, it may send metadata to the computer 600 identifying the piece of equipment, for example, a UV Light, a Gas Vaporizer, and/or an air purifier and an administer may assign that piece of equipment to a space or the system may be configured to automatically assign that piece of equipment to a space. FIG. 8 is yet another example of a database where the equipment is associated with a room in a database. For example, as shown in FIG. 8. A first UV light 100-1 may be associated with room 1000-1 and a second UV light 100-2 may be associated with room 1000-2.

In example embodiments, an administrator may have a computer interface, for example, a smart phone or a tablet, which may send commands to the processor 610. For example, the administrator may issue a command, via the interface, to the processor 610 to clean a certain room. For example, room 1000-3. The microprocessor 610 may look to the aforementioned tables to determine what types of equipment are available in room 1000-3 and then initiate decontamination of room 1000-3 based on the scripts pertinent to the type of equipment. In the present example, the microprocessor 610 would control each of the UV light 100, gas vaporizer 200, and air purifier 300 in room 1000-3 either sequentially, in parallel, or a hybrid of both (depending on the preference of the administrator) to disinfect room 1000-3. In the alternative, the administrator may elect to have a plurality of rooms disinfected, for example, all of the rooms in space 1 to be disinfected, rather than a single room. If this is the case, the controller 610 would control each of the equipment 100, 200, and 300 in rooms 1000-1, 1000-2, 1000-3, and 1000-4 to disinfect the rooms 1000-1, 1000-2, 1000-3, and 1000-4.

In example embodiments additional features may be found to be included in the inventive concepts. For example, a sensor 530 may be installed in each room to detect whether a person is present in a room. In example embodiments, if the sensor 530 detects a person in a room, the microprocessor 610 may determine the room should not be disinfected. In addition to being unoccupied, the microprocessor 610 may require that other predisinfecting conditions be satisfied. For example, the disinfecting process may require a manual activation before the processor 610 can execute operations to disinfect a room. As yet another example, the disinfecting operations may be constrained by certain time periods which may be predetermined by a user. For example, a disinfecting operation may only be allowed during certain times of the day. As yet another example, the doors associated with the rooms may include locks which are operatively connected to the processor 610 so that the processor 610 can control the doors to lock and remain locked during a disinfecting operation to prevent people from entering the room while the room is being disinfected. As yet another example, an indicator, for example, a blinking light and/or red light arranged outside of the room may be operatively connected to the processor 610 and the processor 610 may control the indicator to operate during a disinfecting operation to warn people the room is undergoing a disinfecting operation. Example embodiments, of course, anticipate other arrangements. For example, in another nonlimiting example embodiment a sensor may be provided which indicates whether a door is open or becomes opened. For example, the sensor may take the form of an electrical contract having one trace connected to a door frame and another connected to a door so that if the door is swung open the sensor would detect someone entering the room and the processor 610 would use this data to shut off any decontamination operations occurring in that room. In this latter embodiment, the processor 610 may execute disinfecting operations without having a door be required to be locked. In another embodiment, the sensor may include a magnet which may be in one of the door and a door frame and a circuit in the other of the other of the door and the door frame. In this version, the magnet is magnetically coupled to the circuit so that as the circuit is moved from the magnet, for example, by opening the door, the circuit sends a signal to the controller 600 indicating the door is open and the controller 600 may use this signal to cease any active decontamination operations.

By way of example only, an administrator may request that a room be cleaned by entering the request from a computer interface (for example, a wall control, a table, a desk top computer, or a smart phone) to processor 610. The computer interface may be provided outside of the room to be cleaned or inside the room to be cleaned. The request may be sent wirelessly, over a wire, or through a network. Regardless, the inventive concepts anticipate many ways in which the processor 610 may receive the message from the computer interface. The processor 610 may check to ensure the room is not occupied by using data from the aforementioned sensor. If the room is not occupied and the request was made during an authorized time period, the processor 610 may cause the doors of the room to lock and may turn on an indicator so people understand the room is being disinfected. The indicator may be a blinking light, a light emitting a certain color, for example, red, a sound, and/or a message sent to the administrator and the indicator may be placed in the vicinity of the room. The processor 610 may then activate one or more decontamination apparatuses (example, UV light, gas vaporizer, air purifier) to decontaminate the room. The processor 610 may end operations of the decontamination equipment when a timer ends or a target dosage is met. The dosage may be indicated by one or more sensors that may be in a room, for example, a light sensor 500 (which may measure light intensity at an appropriate wavelength) or a chemical sensor 510 that may be placed in the room, for example, under a bed, under a desk, or wherever dosages need to be confirmed. The decontamination apparatuses may be operated simultaneously or sequentially or a combination of the two. For example, rooms that have each of a UV light, a gas vaporizer, and an air purifier may have the UV light operated first and then terminated followed by the gas vaporizer operated and then terminated followed by the air purifier operated and then terminated. In the alternative, all three apparatuses may be run at once. As yet another alternative, two of the apparatuses may be run simultaneously while the third apparatus is run either before the other two apparatus are run or afterwards. When the decontamination operations end the processor 610 may cause the doors to unlock and the room indicator change to show the room is decontaminated. For example, the indicator may generate a green light or may not generate a light at all when decontamination is complete. When decontaminated, the processor 610 may send a signal to the administrator that the room is decontaminated. In the event the door on the room is opened after decontamination, the processor 610 may cause the indicator to provide an indication the room has been compromised, for example, by generating a yellow light or providing a warning to the operator that the room is compromised.

The inventive concepts described herein can be implemented in many ways. For example, the inventive concepts cover a system which includes a server having an electronic memory which associates decontamination equipment with rooms the decontamination equipment may be installed in. In this example, a user may request a room be decontaminated and the processor would inspect the data in the electronic memory as well as scripts that may be in the electronic memory to determine which decontamination equipment should be turned on and how it should be controlled. On the other hand, the rooms may have a control panel which connect to a server and the control panel may have information regarding the type of equipment in the room and control information. In this latter embodiment a user would send a request to a controller to clean a certain room and the controller would send this information to the control panel in the appropriate room and the room's control panel would thereafter control the decontamination operations. As yet another method, each room may include a control panel which may control multiple decontamination equipment in a room and a user may use the control panel to control room decontamination. In this latter embodiment, the user may utilize an interface built into the controller or a wireless device configured to wirelessly connect to the interface to control the control panel.

Other modifications to example embodiments fall within the inventive concepts. For example, in any one of the preceding examples, the systems may be modified to include a sensor outside a space undergoing decontamination. The sensor may be used to detect a person approaching the space undergoing decontamination. For purposes of illustration only, the sensor may be a motion sensor or a thermal sensor placed on a wall or ceiling that allows entry to the space, or a ceiling near the door. As yet another example, the sensor may be integrated with a door handle or a door lock such that a mere touching of the door handle or lock is enough to sense a person near the space. Regardless, the sensor, upon sensing a person approaching the space undergoing decontamination may send information to a controller controlling a decontamination method and the controller may stop the decontamination in order to ensure the person approaching the space is not harmed by the decontamination process. In the alternative, the sensor may be connected to a sound emitter which may emit a warning message to the person approaching the space.

Other modifications to example embodiments fall within the inventive concepts. For example, in any one of the preceding examples, the systems may be modified to include a sensor outside a space undergoing decontamination. The sensor may be used to detect a person approaching the space. For purposes of illustration only, the sensor may be a motion sensor or a thermal sensor placed on a wall or ceiling near a door that allows entry to the space. As yet another example, the sensor may be integrated with a door handle or a door lock such that a mere touching of the door handle or lock is enough to sense a person near the space. Regardless, the sensor, upon sensing a person approaching the space undergoing decontamination may send information to a controller controlling a decontamination method and the controller upon receiving this information may stop the decontamination operations in order to ensure the person approaching the space is not harmed by the decontamination process. An advantage of such a sensor is that because the systems may be configured to shut off when a person approaching a space is detected, door locks, or a control thereof, may not be required.

As yet another modification it is known that UV light is not visible. Thus, when the UV light is used in a decontamination operation and a person is present in the space where the decontamination operation is going on, the person may not know they are being subject to UV light. In order to warn a person they are in the presence of UV light the systems may include a warning system, for example, a light that emits a visible color such as, but not limited to red, magenta, or yellow, in the space undergoing decontamination to warn the person that UV light is being emitted in their space.

As yet another modification, door closure sensors 700 may be incorporated into any of the aforementioned systems and the controllers controlling the decontamination procedures may be configured so that decontamination/sanitizing operations can commence or be executed only if the doors are closed.

As yet another modification, the systems may include one or more people counting sensors 800 to detect whether or not a person is in a room and, if so, prevent the decontamination/sanitizing procedure from commencing or operating. Similarly, the rooms may include sensors configured to detect a presence of a person, for example, thermal sensors or motion sensors to detect whether a person is in a room and, if so, prevent the decontamination/sanitizing procedure from commencing or operating if a person is detected as being in a room undergoing, or is scheduled to undergo, decontamination/sanitizing operations. Such a sensor may obviate the need for a door lock for purposes of decontamination/sanitizing. Such sensors may periodically scan a room to detect whether a person is in the room or may be embodied as a sensor which monitors entry/occupancy of a room. Such sensors may also be embodied as motion sensors which detect a person entering or moving around in a room.

In some situations sensors that detect the presence of a person or occupation of a room may be based on motion detection. However, if a person in a room does not move the sensor may not detect the presence of the person in a room. To compensate for this, example embodiments envision a system where controller 600 does not execute any decontamination operations until after a predetermined period of time (for example, two minutes) from which no motion or occupancy was detected. This feature allows added safety to the room decontamination process. In the event a person is detected in a room and after a decontamination operation in the room commences, the controller 600 would cause the decontamination equipment to cease executing decontamination operations.

As yet another modification the controllers may be configured to perform a countdown before a decontamination/sanitizing operation begins. The countdown may be displayed on a screen at, near, or in an area about to undergo a decontamination/sanitizing operation. In addition, or in the alternative, the countdown may be displayed on a mobile device such as, but not limited to, a cell phone, and i-pad, a laptop computer, or any other device capable of displaying a countdown. In addition, the controller, in lieu of, or in addition to a visual countdown, may cause a noise emitter to generate a noise indicative of the countdown. For example, the noise emitter may generate an audible countdown so a person can hear the countdown in lieu of, or in addition to, seeing the countdown displayed on a screen. Rather than an audible countdown, the noise emitter may generate a noise indicative of the countdown, for example, a noise that increases in volume as the decontamination/sanitizing operation is about to begin. In addition, the noise emitter may also be configured to and generate audible messages so that people in the vicinity of a room about to undergo decontamination/sanitizing or undergoing decontamination/sanitizing operations will clearly understand what operations will take place or are taking place.

As yet another modification, the decontamination/sanitizing systems may have various lights 900 incorporated therein which indicate a decontamination/sanitizing is operating or is about to operate. The lights 900, for example, may be turned on, and potentially flashing during a countdown before a decontamination/sanitizing begins and may be steady when the decontamination/sanitizing operations are underway. The light, for example, may be purple in color. The lights 900, in one embodiment, may be incorporated in the decontamination/sanitizing equipment, for example, any one of or all of, the UV light 100, the gas vaporizer 200, and the air purifier 300. In the alternative, the light may be separate from anyone of the UV light 100, the gas vaporizer 200, and the air purifier 300 and may be placed in a room or outside of a room ready to be or is undergoing a decontamination/sanitizing operation.

As yet another modification, each piece of decontamination equipment, for example, the UV light 100, the gas vaporizer 200, and the air purifier 300 may be connected to a node 2000, for example, a PoE node, that controls operation of the decontamination equipment. The node 2000 may be operatively connected to a controller through a network or some other means. Each of the decontamination equipment 100, 200, and 300 may have their own node 2000 or may be controlled by a common node 2000. Regardless, the node 2000 may be configured to receive an expected signal, for example, a heartbeat signal, and the node may use this expected signal to keep the equipment operating. In this embodiment, if the expected signal is not received when expected, for example, within an expected time period, the node(s) 2000 would shut down the decontamination equipment 100, 200, and 300. This embodiment may be important for various reasons. For example, if a server goes offline or a network shuts down and it is desired to turn off or shutdown the decontamination/sanitizing operations, the decontamination equipment 100, 200, 300 may not receive a command to shut down. However, when this safety feature is built into the node(s) 2000 such that when the node(s) 2000 does not receive an expected signal, the node(s) 2000 automatically controls the decontamination equipment to shut down or be prevented from operating.

As yet another modification, the system may be configured so that a user can control a level of room decontamination/sanitizing. For example, under certain circumstances it may be determined that an acceptable level of decontamination for one room is 99% whereas other rooms require a deeper level of decontamination/sanitizing, for example, 99.9%. Thus, inventor's system is flexible enough to a user to adjust a level of a decontamination/sanitation for one or more rooms or spaces.

As yet another modification, LED lighting strips may be added to fixture, for example, a fixture of the UV Light 100. The LED lighting strips may provide general illumination and may offer multiple colors or a white light Kelvin levels. As such, the LED lighting strips may provide a controllable range of color or white-tunable light. The control of the LED lighting strips may be implemented by a node attached to the UV Light 100 or a controller 600 operatively connected to the UV Light 100.

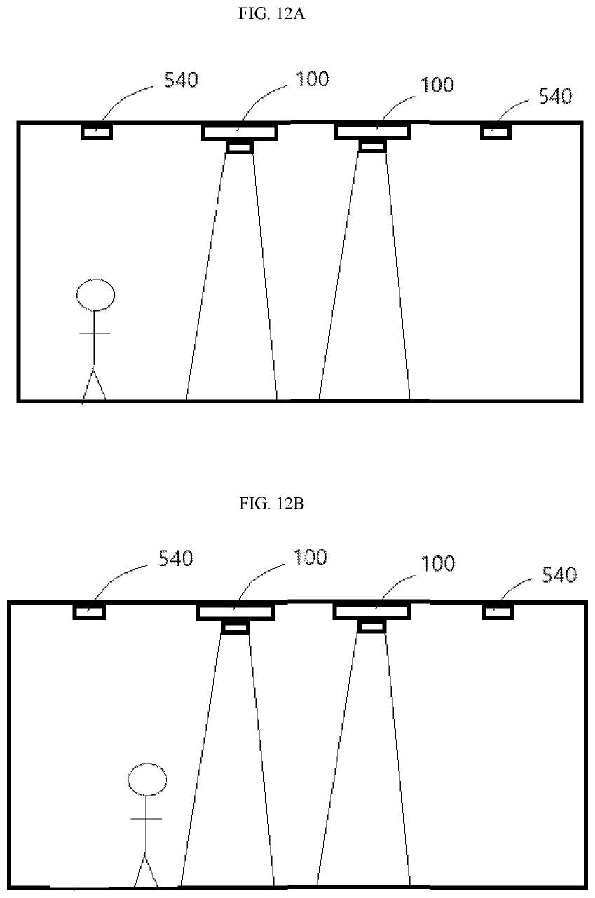

FIGS. 12A and 12B illustrate another aspect of example embodiments. In FIG. 12A two UV lights 100 are illustrated projecting UV light downwards. These UV lights maybe placed in a room or in an open area, for example, a hallway. As one skilled in the art would understand, the UV lights may generally resemble cones of light which may or may not overlap. Persons outside of the “cones” of UV light are generally safe, however, persons who make contact with the “cones” of UV light may be harmed. To this end, detectors 540 configured to sense the presence and/or location of a person near the UV lights 100 may be placed to detect a location of a person and the controller 600 may use this data to control the UV lights 100. For example, so long as a person remains far enough from the cones of UV light, as illustrated in FIG. 12A the UV lights 100 may continue to generate UV light and disinfect the surfaces they are intended to disinfect. However, if the detectors 540 detect a person comes too close to a “cone” of UV light, as shown in FIG. 12B, the controller 600 may cause a UV light 100 to cease generating UV light. The allowable proximity of a person may be predetermined by an owner of the system and may be programmed into controller 600. In a similar vein, gas vaporizers 200, or other disinfecting apparatuses, may be controlled. Like the light example, there may be regions where the gas distribution is so high that it is unsafe for persons to be. Thus, if it is detected that a person is too close to a gas vaporizers 200 the controller 600 may simply turn off said gas vaporizer. Example embodiments anticipate motion sensor(s), people counters, occupancy sensing could be used on the periphery of an open area wherein the open area includes one or more pieces of decontamination equipment. In this case, the decontamination equipment may be controlled by the controller and the decontamination equipment may continue to run so long as those sensors are showing no occupancy in the areas immediately undergoing decontamination.

In addition to the above concepts, the previously described system(s) may further include a touch panel for activating, shutoff, time remaining, and status info (whether the disinfecting operations failed or whether the room is cleaned). Further, the touch panel may log data which may be usable as an audit trail for usage/success statistics and/or maintaining standards & certifications, employee/user assurance, as court evidence perhaps, etc.

What I claim is:

1. A system comprising:
a first piece decontamination equipment configured to decontaminate surfaces in a room;
an electronic database associating the first piece of decontamination equipment with the room;
a processor configured to control the first piece of decontamination equipment by executing a first script;
a computer interface configured to prompt the processor to control the first piece of decontamination equipment by executing the first script.

2. The system of claim 1, wherein the first piece of decontamination equipment is one of an air purifier, a UV light, and a gas vaporizer.

3. The system of claim 1, further comprising:
a second piece of decontamination equipment configured to decontaminate surfaces in the room, wherein the electronic data base associates the second piece of decontamination equipment with the room and the processor is configured to control the second piece of decontamination equipment by executing a second script.

4. The system of claim 3, wherein the processor executes the first and second scripts simultaneously so the first and second pieces of decontamination operate simultaneously.

5. The system of claim 3, wherein the processor executes the first and second scripts sequentially so the first and second pieces of decontamination operate sequentially.

6. The system of claim 3, wherein one of the first and second pieces of decontamination equipment dispenses a chemical and the other dispenses UV light.

7. The system of claim 3, wherein one of the first and second scripts terminates when the processor determines a dosage level was met or exceeded.

8. The system of claim 3, wherein one of the first and second scripts terminates after a predetermined period of time has been met or exceeded.

9. The system of claim 1, wherein the processor is operatively connected to a door lock to lock the door during a decontamination operation.

10. The system of claim 1, wherein the processor is operatively connected to an indicator so that the indicator indicates the first piece of decontamination equipment is operating.

11. A system comprising:
a plurality of decontamination equipment in a plurality of rooms;
an electronic memory associating the plurality of decontamination equipment to a plurality of rooms and storing various scripts for operating the plurality of decontamination equipment;
a microprocessor configured to control the plurality of decontamination equipment using the various scripts; and
a computer interface configured to prompt the processor to control decontamination equipment in a room to decontaminate the room by identifying the decontamination equipment in the room and running the appropriate scripts.

12. The system of claim 11, further comprising:
a plurality of sensors in a plurality of rooms, wherein the electronic memory associates the plurality of sensors with the plurality of rooms.

13. The system of claim 12, wherein the plurality of sensors are configured to detect one of a chemical, UV light, and carbon dioxide.

14. The system of claim 11, wherein the processor is operatively connected to a door to lock the door during a decontamination operation.

15. The system of claim 11, wherein when the computer interface prompts the processor to control decontamination equipment in a room the processor determines which equipment is in the room by inspecting the electronic memory and determines which scripts are associated with the equipment.

16. The system of claim 15, wherein the determined scripts are executed in parallel.

17. The system of claim 15, wherein the determined scripts are executed sequentially

18. The system of claim 15, wherein the processor determines which sensors are in the room being decontaminated and the scripts are terminated after one of a period of time has elapsed and a dosage level is met or exceeded as measured by at least one of the sensors in the room being decontaminated.

19. The system of claim 11, wherein the processor is configured to activate an indicator that indicates a room is being decontaminated when a script is being executed.

20. The system of claim 11, further comprising a sensor to determine whether a person is in a room, wherein the processor does not execute a decontamination script when a person is detected.

21. A system comprising:
a first piece decontamination equipment configured to decontaminate surfaces in an open area;
an electronic database associating the first piece of decontamination equipment with the open area;
a processor configured to control the first piece of decontamination equipment by executing a first script;
a computer interface configured to prompt the processor to control the first piece of decontamination equipment by executing the first script; and
at least one detector configured to sense one or more people at a periphery around the open area, the periphery being found a safe area where people can stay without suffering an ill effect from the first piece of decontamination equipment.

22. The system of claim 21, wherein the first piece of decontamination equipment is one of an air purifier, a UV light, and a gas vaporizer.

23. The system of claim 21, further comprising:
a second piece of decontamination equipment configured to decontaminate surfaces in the open area, wherein the electronic data base associates the second piece of decontamination equipment with the open area and the processor is configured to control the second piece of decontamination equipment by executing a second script.

24. The system of claim 23, wherein the processor executes the first and second scripts simultaneously so the first and second pieces of decontamination operate simultaneously.

25. The system of claim 23, wherein the processor executes the first and second scripts sequentially so the first and second pieces of decontamination operate sequentially.

26. The system of claim 23, wherein one of the first and second pieces of decontamination equipment dispenses a chemical and the other dispenses UV light.

27. The system of claim 23, wherein one of the first and second scripts terminates when the processor determines a dosage level was met or exceeded or when a person is detected to be within the periphery around the open area.

28. The system of claim 23, wherein one of the first and second scripts terminates after a predetermined period of time has been met or exceeded.

29. The system of claim 1, wherein the processor is operatively connected to an indicator so that the indicator indicates the first piece of decontamination equipment is operating.
